(19) United States
(12) Patent Application Publication
(10) Pub. No.: US 2026/0335873 A1
(43) Pub. Date: Oct. 29, 2026

(54) Ethylene Oxychlorination Catalyst, Preparation Method Thereof, and Process for Preparing 1,2-Dichloroethane Through Ethylene Oxychlorination

(71) Applicant: Qingdao University of Science & Technology, Qingdao (CN)

(72) Inventors: Xiaoyan Sun, Qingdao (CN); Yuanyuan Guo, Qingdao (CN); Xuetao Chen, Qingdao (CN); Jun Zhao, Qingdao (CN); Lili Wang, Qingdao (CN); Wenying Zhao, Qingdao (CN); Li Xia, Qingdao (CN); Shuguang Xiang, Qingdao (CN)

(21) Appl. No.: 19/431,598

(22) Filed: Dec. 23, 2025

(30) Foreign Application Priority Data
Apr. 29, 2025 (CN) 202510561219.7

(51) Int. Cl. B01J 29/14; B01J 37/02; B01J 37/04; B01J 37/12; C07C 17/154; C07C 19/045

(52) U.S. Cl. CPC B01J 29/146; B01J 37/0201; B01J 37/04; B01J 37/12; C07C 17/154; C07C 19/045

(57) ABSTRACT

Provided are an ethylene oxychlorination catalyst, a preparation method thereof, and a process for preparing 1,2-dichloroethane through ethylene oxychlorination. The ethylene oxychlorination catalyst includes a composite support and an active component attached to the composite support, where the composite support is produced from γ-Al₂O₃ and a Y-type molecular sieve; and the active component includes copper, with a loading amount of the copper being 3 wt % to 6 wt %.

### CROSS REFERENCE TO RELATED APPLICATION

This patent application claims the benefit and priority of Chinese Patent Application No. 202510561219.7 filed with the China National Intellectual Property Administration on Apr. 29, 2025, the disclosure of which is incorporated by reference herein in its entirety as part of the present application.

### TECHNICAL FIELD

The present disclosure relates to the technical field of catalyst preparation, and in particular relates to an ethylene oxychlorination catalyst, a preparation method thereof, and a process for preparing 1,2-dichloroethane through ethylene oxychlorination.

### BACKGROUND

Ethylene oxychlorination is one of the primary processes for producing 1,2-dichloroethane (also known as ethylene dichloride (EDC)). EDC serves as a crucial intermediate in the production of polyvinyl chloride (PVC). With the growing demand for PVC, the need for EDC, an intermediate for preparing PVC, has also been increasing. The key to improving the efficiency of EDC production lies in enhancing the conversion rate of hydrogen chloride and the selectivity for EDC during an EDC production process. The development of efficient catalysts holds a promising application prospect for improving both the conversion rate of hydrogen chloride and the selectivity for EDC. The conventional catalysts tend to cause the generation of by-products at high temperatures, resulting in resource waste and environmental pollution. The structures and materials of catalysts can be optimized to reduce the generation of by-products and improve the selectivity for target products, thereby meeting environmental protection and energy conservation requirements.

In the prior art, when traditional catalysts are used to catalyze the ethylene oxychlorination, pressurization is required to achieve the high conversion rate of hydrogen chloride and the high selectivity for EDC. However, the pressurization presents high requirements for equipment, and poses some safety risks. Therefore, developing an ethylene oxychlorination catalyst capable of achieving the high conversion rate of hydrogen chloride and the high selectivity for EDC under ambient pressure has a significant economic value and a promising application prospect for the chemical industry.

### SUMMARY

In view of this, an object of the present disclosure is to provide an ethylene oxychlorination catalyst, a preparation method thereof, and a process for preparing 1,2-dichloroethane through ethylene oxychlorination. In the present disclosure, the ethylene oxychlorination catalyst can achieve the oxychlorination of ethylene under ambient pressure, with a high conversion rate of hydrogen chloride and a high selectivity for 1,2-dichloroethane.

To achieve the object of the present disclosure, the present disclosure provides the following technical solutions:

The present disclosure provides an ethylene oxychlorination catalyst, including: a composite support and an active component attached to the composite support, where the composite support is produced from γ-Al₂O₃ and a Y-type molecular sieve; and the active component includes copper, with a loading amount of the copper being 3 wt % to 6 wt %.

In some embodiments, the active component further includes an additive, where the additive includes at least one selected from the group consisting of magnesium, cerium, calcium, potassium, and lanthanum, with a loading amount of the additive being 0.5 wt % to 2 wt %.

In some embodiments, a mass ratio of the γ-Al₂O₃ to the Y-type molecular sieve is in a range of 1-2:1.

In some embodiments, the Y-type molecular sieve is selected from the group consisting of a mesoporous Y-type molecular sieve and a microporous Y-type molecular sieve.

The present disclosure also provides a method for preparing the ethylene oxychlorination catalyst as described in the above technical solutions, including the following steps:
mixing the γ-Al₂O₃, the Y-type molecular sieve, and water, and conducting a first impregnation, a first drying, and a first calcination in sequence to obtain the composite support;
and mixing the composite support and a copper salt solution, and conducting a second impregnation, a second drying, and a second calcination in sequence to obtain the ethylene oxychlorination catalyst.

In some embodiments, mixing the γ-Al₂O₃, the Y-type molecular sieve, and the water is conducted through stirring and an ultrasonic treatment in sequence, and the ultrasonic treatment is conducted for 20 min to 40 min;
the first impregnation is conducted for 10 h to 15 h;
the first drying is conducted at a temperature of 100 °C to 130 °C; and
the first calcination is conducted at a temperature of 500 °C to 600 °C in an air atmosphere for 4 h to 6 h.

In some embodiments, a copper salt in the copper salt solution includes at least one selected from the group consisting of copper nitrate and copper chloride; and a mass ratio of the composite support to the copper salt solution based on the copper is in a range of 1:0.03-0.06.

In some embodiments, mixing the composite support and the copper salt solution is conducted through stirring and an ultrasonic treatment in sequence, and the ultrasonic treatment is conducted for 20 min to 40 min;
the second impregnation is conducted for 10 h to 15 h;
the second drying is conducted at a temperature of 100 °C to 130 °C for 10 h to 15 h; and
the second calcination is conducted at a temperature of 500 °C to 600 °C in an air atmosphere for 4 h to 6 h.

The present disclosure also provides a process for preparing 1,2-dichloroethane through ethylene oxychlorination, including the following step:
subjecting ethylene, hydrogen chloride, and oxygen as raw materials to an oxychlorination reaction in the presence of a catalyst to obtain the 1,2-dichloroethane, where
the oxychlorination reaction is conducted under a pressure of 0.1 MPa; and
the catalyst is the ethylene oxychlorination catalyst as described in the above technical solutions or the ethylene oxychlorination catalyst prepared by the method as described in the above technical solutions.

In some embodiments, a flow rate ratio of the ethylene, the hydrogen chloride, and the oxygen is in a range of 1.05-1.1:2:0.6-0.7, a total flow rate of the ethylene, the hydrogen chloride, and the oxygen is 35 mL/min to 38 mL/min, and based on the hydrogen chloride, a gas hourly space velocity is 200 h⁻¹ to 250 h⁻¹;
the oxychlorination reaction is conducted at a temperature of 190 °C to 230 °C; and
the oxychlorination reaction is conducted in a reactor, and the reactor includes a fixed bed reactor or a fluidized bed reactor.

The present disclosure provides an ethylene oxychlorination catalyst.

In the present disclosure, γ-Al₂O₃ and a Y-type molecular sieve are employed as a composite support to replace a single-component support. The composite support has a large specific surface area and abundant pore structures and active sites. The composite support has a unique pore structure, a tunable pH, and excellent thermal stability, which enables the resistance to carbon deposition. The unique pore structure of the composite support enables the uniform distribution of an active component on the composite support, which reduces the agglomeration of the active component and enhances the effective utilization of the active component to maintain the high reactivity. The ethylene oxychlorination catalyst includes the γ-Al₂O₃ and the Y-type molecular sieve as the composite support and copper as the active component. As a result, the ethylene oxychlorination catalyst can achieve the oxychlorination of ethylene even under ambient pressure, with a high conversion rate of hydrogen chloride and a high selectivity for 1,2-dichloroethane. Experimental data shows that, when the ethylene oxychlorination catalyst in the present disclosure is used in ethylene oxychlorination, a conversion rate of hydrogen chloride reaches not less than 99%, and the selectivity for 1,2-dichloroethane is not less than 98%.

Further, the addition of the additive can enhance the dispersion of the active component, and can change the acid-base properties of a surface of the catalyst to slow down the deactivation of active sites, thereby improving the stability of the catalyst.

### BRIEF DESCRIPTION OF THE DRAWINGS

FIG. 1 shows a flow chart of the method for preparing the ethylene oxychlorination catalyst in an embodiment of the present disclosure; and

FIG. 2 shows a flow chart of the device for evaluating the catalyst in an embodiment of the present disclosure.

### DETAILED DESCRIPTION

The present disclosure provides an ethylene oxychlorination catalyst, including: a composite support and an active component attached to the composite support, where
the composite support is produced from γ-Al₂O₃ and a Y-type molecular sieve; and
the active component includes copper, with a loading amount of the copper being 3 wt % to 6 wt %.

In the present disclosure, the ethylene oxychlorination catalyst includes a composite support, where the composite support is produced from γ-Al₂O₃ and a Y-type molecular sieve. In some embodiments of the present disclosure, a mass ratio of the γ-Al₂O₃ to the Y-type molecular sieve is in a range of (1-2):1 and more preferably 1:1, 1.5:1, or 2:1. In some embodiments of the present disclosure, the Y-type molecular sieve is a mesoporous Y-type molecular sieve or a microporous Y-type molecular sieve, more preferably a mesoporous Y-type molecular sieve. The Y-type molecular sieve has a lower non-framework aluminum content than a β molecular sieve. Consequently, during the preparation of the ethylene oxychlorination catalyst, the Y-type molecular sieve is more likely to maintain a favorable pore structure and acid site distribution, reducing the risk of deactivation. Furthermore, to enhance the activity of the ethylene oxychlorination catalyst, the composite support produced from the combination of the Y-type molecular sieve and the γ-Al₂O₃ possesses a unique pore structure, a tunable pH, and excellent thermal stability. These characteristics can effectively delay the accumulation of carbon deposits to improve the activity and stability of the ethylene oxychlorination catalyst and extend the service life of the ethylene oxychlorination catalyst. The composite support enables a conversion rate of not less than 99% for hydrogen chloride and a selectivity of not less than 98% for 1,2-dichloroethane.

In the present disclosure, the ethylene oxychlorination catalyst includes an active component attached to the composite support, where the active component includes copper, with a loading amount of the copper being 3 wt % to 6 wt %, preferably 3 wt %, 4 wt %, 5 wt %, or 6 wt %. In the present disclosure, the loading amount of the copper refers to a percentage of a mass of the copper relative to a mass of the composite support.

In some embodiments of the present disclosure, the active component further includes an additive. In some embodiments, the additive includes at least one selected from the group consisting of magnesium, cerium, calcium, potassium, and lanthanum, and is more preferably lanthanum. In some embodiments of the present disclosure, a loading amount of the additive is 0.5 wt % to 2 wt %, more preferably 0.5 wt %, 1 wt %, 1.5 wt %, or 2 wt %. In the present disclosure, the loading amount of the additive refers to a percentage of a mass of the additive relative to the mass of the composite support. The addition of the additive can enhance the dispersion of the active component, and can change the acid-base properties of a surface of the catalyst to slow down the deactivation of active sites, thereby improving the stability of the catalyst.

Single-component supports are widely used in the research on ethylene oxychlorination. However, the single-component supports enable low catalytic activity, which limits the conversion rate of oxychlorination. In the present disclosure, a Y-type molecular sieve is combined with γ-Al₂O₃ to produce a composite support, and copper is loaded as an active component on the composite support to produce an ethylene oxychlorination catalyst. The ethylene oxychlorination catalyst demonstrates a significantly-enhanced catalytic capability and service life, and high catalytic activity. Such an ethylene oxychlorination catalyst exhibits excellent catalytic performance, and can improve the conversion rate of HCl and the selectivity for EDC.

The present disclosure also provides a method for preparing the ethylene oxychlorination catalyst as described in the above technical solutions, including the following steps:
mixing the γ-Al₂O₃, the Y-type molecular sieve, and water, and conducting a first impregnation, a first drying, and a first calcination in sequence to obtain the composite support; and
mixing the composite support and a copper salt solution, and conducting a second impregnation, a second drying, and a second calcination in sequence to obtain the ethylene oxychlorination catalyst.

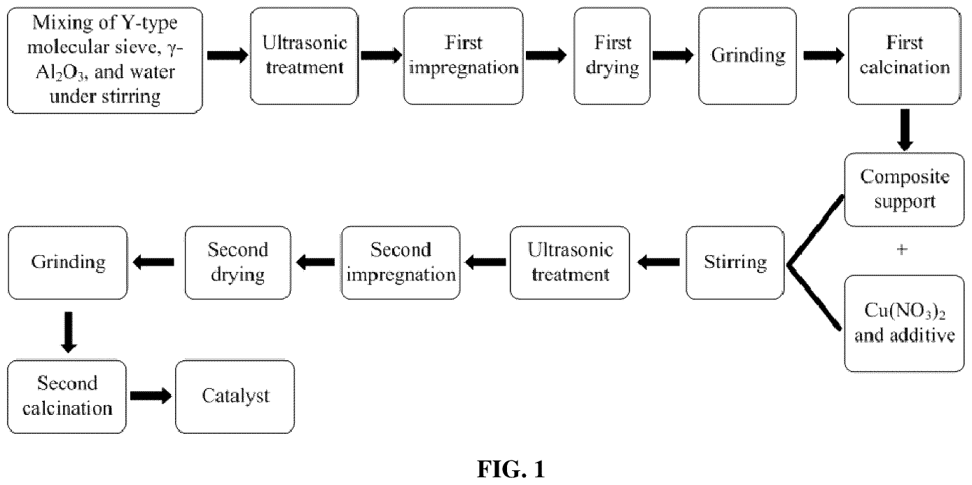

FIG. 1 shows a flow chart of the method for preparing the ethylene oxychlorination catalyst in an embodiment of the present disclosure. The method of the present disclosure will be described below with reference to FIG. 1.

Unless otherwise specified, the raw materials adopted in the present disclosure are preferably all commercially-available products.

In the present disclosure, the γ-Al₂O₃, the Y-type molecular sieve, and the water are mixed, and subjected to the first impregnation, the first drying, and the first calcination in sequence to obtain the composite support.

In some embodiments of the present disclosure, the γ-Al₂O₃ is a powdery γ-Al₂O₃. In a specific embodiment of the present disclosure, a specific surface area of the γ-Al₂O₃ is 144 m²/g.

In some embodiments of the present disclosure, the Y-type molecular sieve is a mesoporous Y-type molecular sieve or a microporous Y-type molecular sieve, and is more preferably a mesoporous Y-type molecular sieve. In some embodiments of the present disclosure, the Y-type molecular sieve is a powdery Y-type molecular sieve. In a specific embodiment of the present disclosure, a specific surface area of the Y-type molecular sieve is 310 m²/g.

In some embodiments of the present disclosure, a mass ratio of the γ-Al₂O₃ to the Y-type molecular sieve is in a range of (1-2):1 and more preferably 1:1, 1.5:1, or 2:1.

In some embodiments of the present disclosure, a ratio of a total mass of the γ-Al₂O₃ and the Y-type molecular sieve to a mass of the water is in a range of 10 g:(25-35) mL and more preferably 10 g:25 mL, 10 g:30 mL, or 10 g:35 mL.

In some embodiments of the present disclosure, mixing the γ-Al₂O₃, the Y-type molecular sieve, and the water is conducted through stirring and an ultrasonic treatment (denoted as a first ultrasonic treatment) in sequence. In some embodiments, the first ultrasonic treatment is conducted for 20 min to 40 min and more preferably 20 min, 30 min, or 40 min. In some embodiments of the present disclosure, the first impregnation is conducted at room temperature, that is, neither additional heating nor additional cooling is required. In some embodiments, the first impregnation is conducted for 10 h to 15 h and more preferably 10 h, 11 h, 12 h, 13 h, 14 h, or 15 h.

In some embodiments of the present disclosure, the first drying is conducted at a temperature of 100 °C to 130 °C and more preferably 100 °C, 105 °C, 110 °C, 115 °C, 120 °C, 125 °C, or 130 °C. In some embodiments, the first drying is conducted for 10 h to 15 h and more preferably 10 h, 11 h, 12 h, 13 h, 14 h, or 15 h. In some embodiments of the present disclosure, the first drying is conducted in a blast drying oven.

In some embodiments, the method further includes, after the first drying and before the first calcination, conducting a first grinding. In the present disclosure, there is no specific limitation on parameters of the first grinding, as long as a blocky material obtained from the first drying can be ground into a powder.

In some embodiments of the present disclosure, the first calcination is conducted at a temperature of 500 °C to 600 °C and more preferably 500 °C, 510 °C, 520 °C, 530 °C, 540 °C, 550 °C, 560 °C, 570 °C, 580 °C, 590 °C, or 600 °C, for 4 h to 6 h and more preferably 4 h, 5 h, or 6 h, and the first calcination is conducted in an air atmosphere.

In the present disclosure, mixing the γ-Al₂O₃, the Y-type molecular sieve, and the water and conducting the first impregnation, the first drying, and the first calcination in sequence can achieve the thorough integration of the γ-Al₂O₃ with the Y-type molecular sieve. Particularly, the first calcination can facilitate the formation of abundant acid sites and promote the subsequent loading of a metal or another component, thereby enhancing the catalytic performance.

In the present disclosure, after the composite support is obtained, the composite support and the copper salt solution are mixed, and subjected to the second impregnation, the second drying, and the second calcination in sequence to obtain the ethylene oxychlorination catalyst.

In some embodiments of the present disclosure, a copper salt in the copper salt solution includes copper nitrate and/or copper chloride, and is more preferably copper nitrate and still more preferably copper nitrate trihydrate (Cu(NO₃)₂·3H₂O).

In some embodiments of the present disclosure, a mass ratio of the composite support to the copper salt solution based on the copper is in a range of 1:(0.03-0.06) and more preferably 1:0.03, 1:0.04, 1:0.05, or 1:0.06.

In some embodiments of the present disclosure, a solvent for the copper salt solution is water and more preferably deionized water. In some embodiments of the present disclosure, based on the copper, a concentration of the copper salt solution is 10 mg/mL to 20 mg/mL and more preferably 10 mg/mL, 13.3 mg/mL, 16.7 mg/mL, or 20 mg/mL.

In some embodiments of the present disclosure, an amount ratio of the copper salt solution to the composite support is in a range of 30 mL:(9-11) g and more preferably 30 mL:9 g, 30 mL:10 g, or 30 mL:11 g.

In some embodiments of the present disclosure, under a condition that the active component of the ethylene oxychlorination catalyst includes the additive, the composite support is impregnated with a copper salt solution including an additive salt. In some embodiments, in the copper salt solution including the additive salt, a mass ratio of the additive to the composite support is in a range of (0.005-0.02):1 and more preferably 0.005:1, 0.01:1, 0.015:1, or 0.02:1. In some embodiments of the present disclosure, under a condition that the additive is lanthanum, the additive salt is lanthanum nitrate and more preferably lanthanum nitrate hexahydrate (La(NO₃)₃·6H₂O). In some embodiments of the present disclosure, an amount ratio of the copper salt solution including the additive salt to the composite support is in a range of 30 mL:(9-11) g and more preferably 30 mL:9 g, 30 mL:10 g, or 30 mL:11 g.

In some embodiments of the present disclosure, mixing the composite support and the copper salt solution is conducted through stirring and an ultrasonic treatment (denoted as a second ultrasonic treatment) in sequence. In some embodiments, the second ultrasonic treatment is conducted for 20 min to 40 min and more preferably 20 min, 30 min, or 40 min.

In some embodiments of the present disclosure, the second impregnation is conducted at room temperature, that is, neither additional heating nor additional cooling is required. In some embodiments, the second impregnation is conducted for 10 h to 15 h and more preferably 10 h, 11 h, 12 h, 13 h, 14 h, or 15 h.

In some embodiments of the present disclosure, the second drying is conducted at a temperature of 100 °C to 130 °C and more preferably 100 °C, 110 °C, 120 °C, or 130 °C. In some embodiments, the second drying is conducted for 10 h to 15 h and more preferably 10 h, 11 h, 12 h, 13 h, 14 h, or 15 h. In some embodiments of the present disclosure, the second drying is conducted in a blast drying oven.

In some embodiments, the method further includes, after the second drying and before the second calcination, conducting a second grinding. In the present disclosure, there is no specific limitation on parameters of the second grinding, as long as a blocky material obtained from the second drying can be ground into a powder.

In some embodiments of the present disclosure, the second calcination is conducted at a temperature of 500 °C to 600 °C and more preferably 500 °C, 510 °C, 520 °C, 530 °C, 540 °C, 550 °C, 560 °C, 570 °C, 580 °C, 590 °C, or 600 °C, for 4 h to 6 h and more preferably 4 h, 5 h, 6 h, and the second calcination is conducted in an air atmosphere. In the present disclosure, the second calcination is conducive to the decomposition of a metal salt into an oxide during a calcination process to produce a metal oxide as a catalytically-active species.

In some embodiments of the present disclosure, the ethylene oxychlorination catalyst is in a powdered form.

The present disclosure also provides a process for preparing 1,2-dichloroethane through ethylene oxychlorination, including the following step:
subjecting ethylene, hydrogen chloride, and oxygen as raw materials to an oxychlorination reaction in the presence of a catalyst to obtain the 1,2-dichloroethane; where
the oxychlorination reaction is conducted under a pressure of 0.1 MPa; and
the catalyst is the ethylene oxychlorination catalyst as described in the above technical solutions or the ethylene oxychlorination catalyst prepared by the method as described in the above technical solutions.

In some embodiments of the present disclosure, the ethylene oxychlorination catalyst is subjected to a pretreatment before use. In some embodiments, the pretreatment includes: tabletting the ethylene oxychlorination catalyst and then sieving to 40 mesh to 60 mesh.

In some embodiments of the present disclosure, the oxychlorination reaction is conducted in a reactor, and the reactor includes a fixed bed reactor or a fluidized bed reactor. In some embodiments of the present disclosure, the fixed bed reactor includes a zirconium tube reactor. In some embodiments, the catalyst is arranged in a middle section of the zirconium tube reactor. In some embodiments, upper and lower sections of the zirconium tube reactor are filled with quartz sand.

In some embodiments, the process further includes, before the ethylene, the hydrogen chloride, and the oxygen are introduced, introducing a protective atmosphere into the reactor. In some embodiments, the protective atmosphere includes nitrogen.

In some embodiments of the present disclosure, a flow rate ratio of the ethylene, the hydrogen chloride, and the oxygen is in a range of (1.05-1.1):2:(0.6-0.7) and more preferably 1.05:2:0.65. In some embodiments, a total flow rate of the ethylene, the hydrogen chloride, and the oxygen is 35 mL/min to 38 mL/min and more preferably 35 mL/min, 36 mL/min, 37 mL/min, or 38 mL/min. In some embodiments, based on the hydrogen chloride, a gas hourly space velocity is 200 h⁻¹ to 250 h⁻¹ and more preferably 200 h⁻¹, 210 h⁻¹, 220 h⁻¹, 230 h⁻¹, 240 h⁻¹, or 250 h⁻¹. In some embodiments of the present disclosure, the ethylene, the hydrogen chloride, and the oxygen are introduced into control pipelines from cylinders through pressure-reducing valves, and then fed at flow rates accurately controlled by precision flow meters into the zirconium tube reactor. In some embodiments, the ethylene, the hydrogen chloride, and the oxygen enter the zirconium tube reactor from a bottom of the zirconium tube reactor, and are heated by the quartz sand in a lower section of the zirconium tube reactor and then come into full contact with the catalyst in a middle section, and subjected to the oxychlorination reaction.

In some embodiments of the present disclosure, the oxychlorination reaction is conducted under a pressure of 0.1 MPa, which is ambient pressure.

In some embodiments of the present disclosure, the oxychlorination reaction is conducted at a temperature of 190 °C to 230 °C and more preferably 190 °C, 200 °C, 210 °C, 220 °C, or 230 °C.

In some embodiments of the present disclosure, after the oxychlorination reaction is completed, a resulting reaction product is tested by gas chromatography to determine the selectivity for EDC. In addition, a gas exiting a top of the zirconium tube reactor is introduced into water to absorb hydrogen chloride, a hydrogen chloride-absorbed solution is titrated with a NaOH solution based on acid-base titration, and a conversion rate of hydrogen chloride is calculated, thereby evaluating the performance of the catalyst.

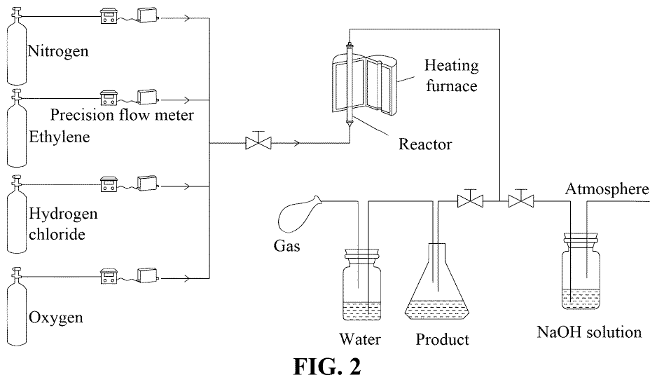

FIG. 2 shows a flow chart of the device for evaluating the catalyst in an embodiment of the present disclosure. Specifically, before the evaluation, nitrogen is introduced to create a nitrogen atmosphere in the reactor. Then, ethylene, hydrogen chloride, and oxygen are introduced, and subjected to an oxychlorination reaction. During the oxychlorination reaction, a left valve is opened and a right valve is closed, such that a gas and a liquid produced are introduced into conical flasks for products. The gas is then introduced into water to remove HCl, and a resulting purified gas is finally collected in a gas bag. To determine a conversion rate of hydrogen chloride, the left valve is closed and the right valve is opened to collect a gas for 2 min to measure the conversion rate of hydrogen chloride.

The ethylene oxychlorination catalyst, the preparation method thereof, and the process for preparing 1,2-dichloroethane through ethylene oxychlorination provided by the present disclosure will be described in detail below with reference to examples, but the examples should not be construed as limiting the scope of the present disclosure.

### Example 1

5 g of a mesoporous Y-type molecular sieve (with a specific surface area of 310 m²/g) and 5 g of γ-Al₂O₃ (with a specific surface area of 144 m²/g) were added to 30 mL of deionized water. A resulting mixture was stirred for thorough mixing, and then subjected to ultrasonic treatment for 30 min. Impregnation was conducted at room temperature for 12 h. A resulting product was subjected to oven-drying in a blast drying oven at 110 °C for 12 h. A resulting dried material was ground into a powder. The powder was then calcined in an air atmosphere at 560 °C for 4 h to obtain a Y-type molecular sieve/γ-Al₂O₃ composite support.

A specific surface area and a pore size of the Y-type molecular sieve/γ-Al₂O₃ composite support were tested by the Brunauer-Emmett-Teller (BET) method. Test results show that the composite support has a specific surface area of 170 m²/g and a pore size of 11 nm.

1.1325 g of Cu(NO₃)₂·3H₂O was dispersed uniformly in 30 mL of deionized water to obtain a copper salt solution. Then, 10 g of the Y-type molecular sieve/γ-Al₂O₃ composite support was added to the copper salt solution. A resulting mixture was stirred for thorough mixing, and then subjected to ultrasonic treatment for 30 min. Impregnation was conducted at room temperature for 12 h. A resulting product was subjected to oven-drying in a blast drying oven at 110 °C for 12 h. A resulting dried material was ground into a powder. The powder was then calcined in an air atmosphere at 560 °C for 4 h to obtain a Cu₃-Y-type molecular sieve/γ-Al₂O₃ composite support catalyst with a loading amount of copper being 3 wt %.

Evaluation of the catalyst: The prepared catalyst was tableted and sieved to 40 mesh to 60 mesh, and then arranged in a fixed bed reactor. Nitrogen was introduced into the fixed bed reactor to create a nitrogen atmosphere. Then, ethylene, hydrogen chloride, and oxygen were introduced. A total flow rate of the ethylene, the hydrogen chloride, and the oxygen was controlled at 37 mL/min. A flow rate ratio of the ethylene, the hydrogen chloride, and the oxygen was controlled at 1.05:2:0.65. Based on the hydrogen chloride, a gas hourly space velocity was 220 h⁻¹. A resulting system was subjected to a reaction at a temperature of 190 °C to 230 °C and a pressure of 0.1 MPa. A resulting reaction product was tested by gas chromatography to determine the selectivity for EDC. A gas exiting a top of the fixed bed reactor was introduced into water to absorb HCl. A resulting HCl-absorbed solution was titrated with a NaOH solution based on acid-base titration, and a conversion rate of HCl was calculated. Specific data results of 7 h and 800 h of operation are shown in Tables 1 and 2, respectively.

### Example 2

5 g of a mesoporous Y-type molecular sieve (with a specific surface area of 310 m²/g) and 5 g of γ-Al₂O₃ (with a specific surface area of 144 m²/g) were added to 30 mL of deionized water. A resulting mixture was stirred for thorough mixing, and then subjected to ultrasonic treatment for 30 min. Impregnation was conducted at room temperature for 12 h. A resulting product was subjected to oven-drying in a blast drying oven at 110 °C for 12 h. A resulting dried material was ground into a powder. The powder was then calcined in an air atmosphere at 560 °C for 4 h to obtain a Y-type molecular sieve/γ-Al₂O₃ composite support.

1.51 g of Cu(NO₃)₂·3H₂O was dispersed uniformly in 30 mL of deionized water to obtain a copper salt solution. Then, 10 g of the Y-type molecular sieve/γ-Al₂O₃ composite support was added to the copper salt solution. A resulting mixture was stirred for thorough mixing, and then subjected to ultrasonic treatment for 30 min. Impregnation was conducted at room temperature for 12 h. A resulting product was subjected to oven-drying in a blast drying oven at 110 °C for 12 h. A resulting dried material was ground into a powder. The powder was then calcined in an air atmosphere at 560 °C for 4 h to obtain a Cu₄-Y-type molecular sieve/γ-Al₂O₃ composite support catalyst with a loading amount of copper being 4 wt %.

Evaluation of the catalyst: The prepared catalyst was tableted and sieved to 40 mesh to 60 mesh, and then arranged in a fixed bed reactor. Nitrogen was introduced into the fixed bed reactor to create a nitrogen atmosphere. Then, ethylene, hydrogen chloride, and oxygen were introduced. A total flow rate of the ethylene, the hydrogen chloride, and the oxygen was controlled at 37 mL/min. A flow rate ratio of the ethylene, the hydrogen chloride, and the oxygen was controlled at 1.05:2:0.65. Based on the hydrogen chloride, a gas hourly space velocity was 220 h⁻¹. A resulting system was subjected to a reaction at a temperature of 190 °C to 230 °C and a pressure of 0.1 MPa. A resulting reaction product was tested by gas chromatography to determine the selectivity for EDC. A gas exiting a top of the fixed bed reactor was introduced into water to absorb HCl. A resulting HCl-absorbed solution was titrated with a NaOH solution based on acid-base titration, and a conversion rate of HCl was calculated. Specific data results of 7 h and 800 h of operation are shown in Tables 1 and 2, respectively.

### Example 3

5 g of a mesoporous Y-type molecular sieve (with a specific surface area of 310 m²/g) and 5 g of γ-Al₂O₃ (with a specific surface area of 144 m²/g) were added to 30 mL of deionized water. A resulting mixture was stirred for thorough mixing, and then subjected to ultrasonic treatment for 30 min. Impregnation was conducted at room temperature for 12 h. A resulting product was subjected to oven-drying in a blast drying oven at 110 °C for 12 h. A resulting dried material was ground into a powder. The powder was then calcined in an air atmosphere at 560 °C for 4 h to obtain a Y-type molecular sieve/γ-Al₂O₃ composite support.

1.8875 g of Cu(NO₃)₂·3H₂O was dispersed uniformly in 30 mL of deionized water to obtain a copper salt solution. Then, 10 g of the Y-type molecular sieve/γ-Al₂O₃ composite support was added to the copper salt solution. A resulting mixture was stirred for thorough mixing, and then subjected to ultrasonic treatment for 30 min. Impregnation was conducted at room temperature for 12 h. A resulting product was subjected to oven-drying in a blast drying oven at 110 °C for 12 h. A resulting dried material was ground into a powder. The powder was then calcined in an air atmosphere at 560 °C for 4 h to obtain a Cu₅-Y-type molecular sieve/γ-Al₂O₃ composite support catalyst with a loading amount of copper being 5 wt %.

Evaluation of the catalyst: The prepared catalyst was tableted and sieved to 40 mesh to 60 mesh, and then arranged in a fixed bed reactor. Nitrogen was introduced into the fixed bed reactor to create a nitrogen atmosphere. Then, ethylene, hydrogen chloride, and oxygen were introduced. A total flow rate of the ethylene, the hydrogen chloride, and the oxygen was controlled at 37 mL/min. A flow rate ratio of the ethylene, the hydrogen chloride, and the oxygen was controlled at 1.05:2:0.65. Based on the hydrogen chloride, a gas hourly space velocity was 220 h⁻¹. A resulting system was subjected to a reaction at a temperature of 190 °C to 230 °C and a pressure of 0.1 MPa. A resulting reaction product was tested by gas chromatography to determine the selectivity for EDC. A gas exiting a top of the fixed bed reactor was introduced into water to absorb HCl. A resulting HCl-absorbed solution was titrated with a NaOH solution based on acid-base titration, and a conversion rate of HCl was calculated. Specific data results of 7 h and 800 h of operation are shown in Tables 1 and 2, respectively.

### Example 4

5 g of a mesoporous Y-type molecular sieve (with a specific surface area of 310 m²/g) and 5 g of γ-Al₂O₃ (with a specific surface area of 144 m²/g) were added to 30 mL of deionized water. A resulting mixture was stirred for thorough mixing, and then subjected to ultrasonic treatment for 30 min. Impregnation was conducted at room temperature for 12 h. A resulting product was subjected to oven-drying in a blast drying oven at 110 °C for 12 h. A resulting dried material was ground into a powder. The powder was then calcined in an air atmosphere at 560 °C for 4 h to obtain a Y-type molecular sieve/γ-Al₂O₃ composite support.

2.265 g of Cu(NO₃)₂·3H₂O was added to 30 mL of deionized water, and a resulting mixture was stirred for thorough mixing to obtain a copper salt solution. Then, 10 g of the Y-type molecular sieve/γ-Al₂O₃ composite support was added to the copper salt solution. A resulting mixture was stirred for thorough mixing, and then subjected to ultrasonic treatment for 30 min. Impregnation was conducted at room temperature for 12 h. A resulting product was subjected to oven-drying in a blast drying oven at 110 °C for 12 h. A resulting dried material was ground into a powder. The powder was then calcined in an air atmosphere at 560 °C for 4 h to obtain a Cu₆-Y-type molecular sieve/γ-Al₂O₃ composite support catalyst with a loading amount of copper being 6 wt %.

Evaluation of the catalyst: The prepared catalyst was tableted and sieved to 40 mesh to 60 mesh, and then arranged in a fixed bed reactor. Nitrogen was introduced into the fixed bed reactor to create a nitrogen atmosphere. Then, ethylene, hydrogen chloride, and oxygen were introduced. A total flow rate of the ethylene, the hydrogen chloride, and the oxygen was controlled at 37 mL/min. A flow rate ratio of the ethylene, the hydrogen chloride, and the oxygen was controlled at 1.05:2:0.65. Based on the hydrogen chloride, a gas hourly space velocity was 220 h⁻¹. A resulting system was subjected to a reaction at a temperature of 190 °C to 230 °C and a pressure of 0.1 MPa. A resulting reaction product was tested by gas chromatography to determine the selectivity for EDC. A gas exiting a top of the fixed bed reactor was introduced into water to absorb HCl. A resulting HCl-absorbed solution was titrated with a NaOH solution based on acid-base titration, and a conversion rate of HCl was calculated. Specific data results of 7 h and 800 h of operation are shown in Tables 1 and 2, respectively.

TABLE 1
Influence of a loading amount of Cu on an HCl conversion
rate and EDC selectivity (reaction time: 7 h)

| No. | | Example 1 | Example 2 | Example 3 | Example 4 |
|---|---|---|---|---|---|
| Loading amount of Cu/% | | 3 | 4 | 5 | 6 |
| HCl | 190 °C | 98.69 | 98.7 | 99.04 | 98.38 |
| Conversion | 200 °C | 98.80 | 99.02 | 99.09 | 98.49 |
| rate/% | 210 °C | 98.86 | 99.25 | 99.31 | 98.74 |
| | 220 °C | 98.77 | 99.07 | 99.16 | 98.72 |
| | 230 °C | 98.48 | 98.7 | 98.74 | 98.34 |
| EDC | 190 °C | 94.86 | 94.92 | 95.03 | 94.25 |
| Selectivity/% | 200 °C | 94.13 | 94.50 | 94.60 | 93.76 |
| | 210 °C | 93.56 | 94.42 | 94.48 | 93.62 |
| | 220 °C | 93.44 | 94.21 | 94.35 | 92.58 |
| | 230 °C | 93.38 | 93.79 | 94.26 | 92.51 |

TABLE 2
Influence of a loading amount of Cu on an HCl conversion
rate and EDC selectivity (reaction time: 800 h)

| No. | | Example 1 | Example 2 | Example 3 | Example 4 |
|---|---|---|---|---|---|
| Loading amount of Cu/% | | 3 | 4 | 5 | 6 |
| HCl | 190 °C | 96.83 | 97.28 | 98.04 | 97.08 |
| Conversion | 200 °C | 96.95 | 98.06 | 98.12 | 97.12 |
| rate/% | 210 °C | 97.61 | 98.87 | 99.05 | 98.02 |
| | 220 °C | 97.46 | 98.43 | 98.86 | 97.33 |
| | 230 °C | 97.34 | 98.02 | 98.11 | 97.31 |
| EDC | 190 °C | 93.47 | 93.57 | 94.04 | 93.28 |
| Selectivity/% | 200 °C | 93.20 | 93.34 | 93.56 | 93.11 |
| | 210 °C | 93.13 | 93.17 | 93.52 | 92.50 |
| | 220 °C | 92.52 | 92.65 | 93.00 | 92.45 |
| | 230 °C | 92.06 | 92.35 | 92.89 | 91.98 |

### Example 5

5 g of a mesoporous Y-type molecular sieve (with a specific surface area of 310 m²/g) and 5 g of γ-Al₂O₃ (with a specific surface area of 144 m²/g) were added to 30 mL of deionized water. A resulting mixture was stirred for thorough mixing, and then subjected to ultrasonic treatment for 30 min. Impregnation was conducted at room temperature for 12 h. A resulting product was subjected to oven-drying in a blast drying oven at 110 °C for 12 h. A resulting dried material was ground into a powder. The powder was then calcined in an air atmosphere at 560 °C for 4 h to obtain a Y-type molecular sieve/γ-Al₂O₃ composite support.

1.8875 g of Cu(NO₃)₂·3H₂O and 0.156 g of La(NO₃)₃·6H₂O were dispersed uniformly in 30 mL of deionized water to produce a salt solution. Then, 10 g of the Y-type molecular sieve/γ-Al₂O₃ composite support was added to the salt solution. A resulting mixture was stirred for thorough mixing, and then subjected to ultrasonic treatment for 30 min. Impregnation was conducted at room temperature for 12 h. A resulting product was subjected to oven-drying in a blast drying oven at 110 °C for 12 h. A resulting dried material was ground into a powder. The powder was then calcined in an air atmosphere at 560 °C for 4 h to obtain a La_{0.5}Cu₅-Y-type molecular sieve/γ-Al₂O₃ composite support catalyst with a loading amount of copper being 5 wt % and a loading amount of lanthanum being 0.5 wt %.

Evaluation of the catalyst: The prepared catalyst was tableted and sieved to 40 mesh to 60 mesh, and then arranged in a fixed bed reactor. Nitrogen was introduced into the fixed bed reactor to create a nitrogen atmosphere. Then, ethylene, hydrogen chloride, and oxygen were introduced. A total flow rate of the ethylene, the hydrogen chloride, and the oxygen was controlled at 37 mL/min. A flow rate ratio of the ethylene, the hydrogen chloride, and the oxygen was controlled at 1.05:2:0.65. Based on the hydrogen chloride, a gas hourly space velocity was 220 h⁻¹. A resulting system was subjected to a reaction at a temperature of 190 °C to 230 °C and a pressure of 0.1 MPa. A resulting reaction product was tested by gas chromatography to determine the selectivity for EDC. A gas exiting a top of the fixed bed reactor was introduced into water to absorb HCl. A resulting HCl-absorbed solution was titrated with a NaOH solution based on acid-base titration, and a conversion rate of HCl was calculated. Specific data results of 7 h and 800 h of operation are shown in Tables 3 and 4, respectively.

### Example 6

5 g of a mesoporous Y-type molecular sieve (with a specific surface area of 310 m²/g) and 5 g of γ-Al₂O₃ (with a specific surface area of 144 m²/g) were added to 30 mL of deionized water. A resulting mixture was stirred for thorough mixing, and then subjected to ultrasonic treatment for 30 min. Impregnation was conducted at room temperature for 12 h. A resulting product was subjected to oven-drying in a blast drying oven at 110 °C for 12 h. A resulting dried material was ground into a powder. The powder was then calcined in an air atmosphere at 560 °C for 4 h to obtain a Y-type molecular sieve/γ-Al₂O₃ composite support.

1.8875 g of Cu(NO₃)₂·3H₂O and 0.312 g of La(NO₃)₃·6H₂O were dispersed uniformly in 30 mL of deionized water to produce a salt solution. Then, 10 g of the Y-type molecular sieve/γ-Al₂O₃ composite support was added to the salt solution. A resulting mixture was stirred for thorough mixing, and then subjected to ultrasonic treatment for 30 min. Impregnation was conducted at room temperature for 12 h. A resulting product was subjected to oven-drying in a blast drying oven at 110 °C for 12 h. A resulting dried material was ground into a powder. The powder was then calcined in an air atmosphere at 560 °C for 4 h to obtain a La₁Cu₅-Y-type molecular sieve/γ-Al₂O₃ composite support catalyst with a loading amount of copper being 5 wt % and a loading amount of lanthanum being 1 wt %.

Evaluation of the catalyst: The prepared catalyst was tableted and sieved to 40 mesh to 60 mesh, and then arranged in a fixed bed reactor. Nitrogen was introduced into the fixed bed reactor to create a nitrogen atmosphere. Then, ethylene, hydrogen chloride, and oxygen were introduced. A total flow rate of the ethylene, the hydrogen chloride, and the oxygen was controlled at 37 mL/min. A flow rate ratio of the ethylene, the hydrogen chloride, and the oxygen was controlled at 1.05:2:0.65. Based on the hydrogen chloride, a gas hourly space velocity was 220 h⁻¹. A resulting system was subjected to a reaction at a temperature of 190 °C to 230 °C and a pressure of 0.1 MPa. A resulting reaction product was tested by gas chromatography to determine the selectivity for EDC. A gas exiting a top of the fixed bed reactor was introduced into water to absorb HCl. A resulting HCl-absorbed solution was titrated with a NaOH solution based on acid-base titration, and a conversion rate of HCl was calculated. Specific data results of 7 h and 800 h of operation are shown in Tables 3 and 4, respectively.

### Example 7

5 g of a mesoporous Y-type molecular sieve (with a specific surface area of 310 m²/g) and 5 g of γ-Al₂O₃ (with a specific surface area of 144 m²/g) were added to 30 mL of deionized water. A resulting mixture was stirred for thorough mixing, and then subjected to ultrasonic treatment for 30 min. Impregnation was conducted at room temperature for 12 h. A resulting product was subjected to oven-drying in a blast drying oven at 110 °C for 12 h. A resulting dried material was ground into a powder. The powder was then calcined in an air atmosphere at 560 °C for 4 h to obtain a Y-type molecular sieve/γ-Al₂O₃ composite support.

1.8875 g of Cu(NO₃)₂·3H₂O and 0.468 g of La(NO₃)₃·6H₂O were dispersed uniformly in 30 mL of deionized water to produce a salt solution. Then, 10 g of the Y-type molecular sieve/γ-Al₂O₃ composite support was added to the salt solution. A resulting mixture was stirred for thorough mixing, and then subjected to ultrasonic treatment for 30 min. Impregnation was conducted at room temperature for 12 h. A resulting product was subjected to oven-drying in a blast drying oven at 110 °C for 12 h. A resulting dried material was ground into a powder. The powder was then calcined in an air atmosphere at 560 °C for 4 h to obtain a La_{1.5}Cu₅-Y-type molecular sieve/γ-Al₂O₃ composite support catalyst with a loading amount of copper being 5 wt % and a loading amount of lanthanum being 1.5 wt %.

Evaluation of the catalyst: The prepared catalyst was tableted and sieved to 40 mesh to 60 mesh, and then arranged in a fixed bed reactor. Nitrogen was introduced into the fixed bed reactor to create a nitrogen atmosphere. Then, ethylene, hydrogen chloride, and oxygen were introduced. A total flow rate of the ethylene, the hydrogen chloride, and the oxygen was controlled at 37 mL/min. A flow rate ratio of the ethylene, the hydrogen chloride, and the oxygen was controlled at 1.05:2:0.65. Based on the hydrogen chloride, a gas hourly space velocity was 220 h⁻¹. A resulting system was subjected to a reaction at a temperature of 190 °C to 230 °C and a pressure of 0.1 MPa.

A resulting reaction product was tested by gas chromatography to determine the selectivity for EDC. A gas exiting a top of the fixed bed reactor was introduced into water to absorb HCl. A resulting HCl-absorbed solution was titrated with a NaOH solution based on acid-base titration, and a conversion rate of HCl was calculated. Specific data results of 7 h and 800 h of operation are shown in Tables 3 and 4, respectively.

### Example 8

5 g of a mesoporous Y-type molecular sieve (with a specific surface area of 310 m²/g) and 5 g of γ-Al₂O₃ (with a specific surface area of 144 m²/g) were added to 30 mL of deionized water. A resulting mixture was stirred for thorough mixing, and then subjected to ultrasonic treatment. Impregnation was conducted at room temperature for 12 h. A resulting product was subjected to oven-drying in a blast drying oven at 110 °C for 12 h. A resulting dried material was then calcined in an air atmosphere at 560 °C for 4 h to obtain a Y-type molecular sieve/γ-Al₂O₃ composite support.

1.8875 g of Cu(NO₃)₂·3H₂O and 0.623 g of La(NO₃)₃·6H₂O were dispersed uniformly in 30 mL of deionized water, and 10 g of the Y-type molecular sieve/γ-Al₂O₃ composite support was added thereto. A resulting mixture was stirred for thorough mixing, and then subjected to ultrasonic treatment. Impregnation was conducted at room temperature for 12 h. A resulting product was subjected to oven-drying in a blast drying oven at 110 °C for 12 h. A resulting dried material was then calcined in an air atmosphere at 560 °C for 4 h to obtain a La₂Cu₅-Y-type molecular sieve/γ-Al₂O₃ composite support catalyst with a loading amount of copper being 5 wt % and a loading amount of lanthanum being 2 wt %.

Evaluation of the catalyst: The prepared catalyst was tableted and sieved to 40 mesh to 60 mesh, and then arranged in a fixed bed reactor. Nitrogen was introduced into the fixed bed reactor to create a nitrogen atmosphere. Then, ethylene, hydrogen chloride, and oxygen were introduced. A total flow rate of the ethylene, the hydrogen chloride, and the oxygen was controlled at 37 mL/min. A flow rate ratio of the ethylene, the hydrogen chloride, and the oxygen was controlled at 1.05:2:0.65. Based on the hydrogen chloride, a gas hourly space velocity was 220 h⁻¹. A resulting system was subjected to a reaction at a temperature of 190 °C to 230 °C and a pressure of 0.1 MPa. A resulting reaction product was tested by gas chromatography to determine the selectivity for EDC. A gas exiting a top of the fixed bed reactor was introduced into water to absorb HCl. A resulting HCl-absorbed solution was titrated with a NaOH solution based on acid-base titration, and a conversion rate of HCl was calculated. Specific data results of 7 h and 800 h of operation are shown in Tables 3 and 4, respectively.

TABLE 3
Influence of an additive content on an HCl conversion
rate and EDC selectivity (reaction time: 7 h)

| No. | | Example 3 | Example 5 | Example 6 | Example 7 | Example 8 |
|---|---|---|---|---|---|---|
| Additive content/% | | 0 | 0.5 | 1 | 1.5 | 2 |
| HCl | 190 °C | 99.04 | 99.06 | 99.09 | 99.15 | 98.38 |
| Conversion | 200 °C | 99.09 | 99.13 | 99.16 | 99.23 | 98.49 |
| rate/% | 210 °C | 99.31 | 99.32 | 99.35 | 99.61 | 98.74 |
| | 220 °C | 99.16 | 99.18 | 99.19 | 99.22 | 98.72 |
| | 230 °C | 98.74 | 98.86 | 98.92 | 98.94 | 98.34 |
| EDC | 190 °C | 95.03 | 96.16 | 97.28 | 98.71 | 97.19 |
| Selectivity/% | 200 °C | 94.60 | 95.98 | 97.15 | 98.48 | 96.99 |
| | 210 °C | 94.48 | 95.79 | 96.39 | 98.03 | 96.74 |
| | 220 °C | 94.35 | 95.76 | 96.00 | 97.17 | 96.09 |
| | 230 °C | 94.26 | 95.62 | 95.88 | 96.25 | 95.81 |

TABLE 4
Influence of an additive content on an HCl conversion
rate and EDC selectivity (reaction time: 800 h)

| No. | | Example 3 | Example 5 | Example 6 | Example 7 | Example 8 |
|---|---|---|---|---|---|---|
| Additive content/% | | 0 | 0.5 | 1 | 1.5 | 2 |
| HCl | 190 °C | 98.04 | 98.08 | 98.11 | 98.13 | 97.21 |
| Conversion | 200 °C | 98.12 | 98.16 | 98.27 | 98.59 | 97.69 |
| rate/% | 210 °C | 99.05 | 99.07 | 99.10 | 99.11 | 98.02 |
| | 220 °C | 98.86 | 98.88 | 98.89 | 98.90 | 98.00 |
| | 230 °C | 98.11 | 98.36 | 98.85 | 98.87 | 97.94 |
| EDC | 190 °C | 94.04 | 96.10 | 96.24 | 97.02 | 96.00 |
| Selectivity/% | 200 °C | 93.56 | 95.72 | 95.92 | 96.53 | 95.88 |
| | 210 °C | 93.52 | 95.64 | 95.82 | 96.48 | 95.76 |
| | 220 °C | 93.00 | 95.56 | 95.80 | 96.27 | 95.41 |
| | 230 °C | 92.89 | 94.92 | 95.27 | 96.08 | 94.87 |

### Comparative Example 1

1.8875 g of Cu(NO₃)₂·3H₂O and 0.468 g of La(NO₃)₃·6H₂O were dispersed uniformly in 30 mL of deionized water, and then, 10 g of γ-Al₂O₃ (with a specific surface area of 144 m²/g) was added thereto. A resulting mixture was stirred for thorough mixing, and then subjected to ultrasonic treatment. Impregnation was conducted at room temperature for 12 h. A resulting product was subjected to oven-drying in a blast drying oven at 110 °C for 12 h. A resulting dried material was then calcined in an air atmosphere at 560 °C for 4 h to obtain a La_{1.5}Cu₅/γ-Al₂O₃ catalyst with a loading amount of copper being 5 wt % and a loading amount of lanthanum being 1.5 wt %.

Evaluation of the catalyst: The prepared catalyst was tableted and sieved to 40 mesh to 60 mesh, and then arranged in a fixed bed reactor. Then, C₂H₄, HCl, and O₂ were introduced. A total flow rate of the C₂H₄, the HCl, and the O₂ was controlled at 37 mL/min. A flow rate ratio of the C₂H₄, the HCl, and the O₂ was controlled at 1.05:2:0.65. Based on the hydrogen chloride, a gas hourly space velocity was 220 h⁻¹. A resulting system was subjected to a reaction at a temperature of 190 °C to 230 °C and a pressure of 0.1 MPa. A resulting reaction product was tested by gas chromatography to determine the selectivity for EDC. A gas exiting a top of the fixed bed reactor was introduced into water to absorb HCl. A resulting HCl-absorbed solution was titrated with a NaOH solution based on acid-base titration, and a conversion rate of HCl was calculated. Specific data results of 7 h and 800 h of operation are shown in Tables 5 and 6, respectively.

### Comparative Example 2

1.8875 g of Cu(NO₃)₂·3H₂O and 0.468 g of La(NO₃)₃·6H₂O were dispersed uniformly in 30 mL of deionized water, and then, 10 g of a mesoporous Y-type molecular sieve (with a specific surface area of 310 m²/g) was added thereto. A resulting mixture was stirred for thorough mixing, and then subjected to ultrasonic treatment. Impregnation was conducted at room temperature for 12 h. A resulting product was subjected to oven-drying in a blast drying oven at 110 °C for 12 h. A resulting dried material was then calcined in an air atmosphere at 560 °C for 4 h to obtain a La_{1.5}Cu₅/Y-type molecular sieve catalyst with a loading amount of copper being 5 wt % and a loading amount of lanthanum being 1.5 wt %.

Evaluation of the catalyst: The prepared catalyst was tableted and sieved to 40 mesh to 60 mesh, and then arranged in a fixed bed reactor. Then, C₂H₄, HCl, and O₂ were introduced. A total flow rate of the C₂H₄, the HCl, and the O₂ was controlled at 37 mL/min. A flow rate ratio of the C₂H₄, the HCl, and the O₂ was controlled at 1.05:2:0.65. Based on the hydrogen chloride, a gas hourly space velocity was 220 h⁻¹. A resulting system was subjected to a reaction at a temperature of 190 °C to 230 °C and a pressure of 0.1 MPa. A resulting reaction product was tested by gas chromatography to determine the selectivity for EDC. A gas exiting a top of the fixed bed reactor was introduced into water to absorb HCl. A resulting HCl-absorbed solution was titrated with a NaOH solution based on acid-base titration, and a conversion rate of HCl was calculated. Specific data results of 7 h and 800 h of operation are shown in Tables 5 and 6, respectively.

TABLE 5
Influence of different supports on an HCl conversion
rate and EDC selectivity (reaction time: 7 h)

| | | | Comparative | Comparative |
|---|---|---|---|---|
| No. | | Example 7 | Example 1 | Example 2 |
| Support type | | La_{1.5}Cu₅—Y-type | La_{1.5}Cu₅/γ-Al₂O₃ | La_{1.5}Cu₅—Y-type |
| | | molecular | | molecular sieve |
| | | sieve/γ-Al₂O₃ | | |
| HCl | 190 °C | 99.15 | 95.89 | 97.55 |
| conversion | 200 °C | 99.23 | 97.11 | 98.04 |
| rate/% | 210 °C | 99.61 | 98.44 | 98.64 |
| | 220 °C | 99.22 | 97.95 | 98.29 |
| | 230 °C | 98.94 | 97.38 | 98.08 |
| EDC | 190 °C | 98.71 | 91.91 | 95.23 |
| selectivity/% | 200 °C | 98.48 | 91.63 | 94.91 |
| | 210 °C | 98.03 | 91.57 | 94.72 |
| | 220 °C | 97.17 | 91.39 | 94.48 |
| | 230 °C | 96.25 | 90.67 | 94.35 |

TABLE 6
Influence of different supports on an HCl conversion
rate and EDC selectivity (reaction time: 800 h)

| | | | Comparative | Comparative |
|---|---|---|---|---|
| No. | | Example 7 | Example 1 | Example 2 |
| Support type | | La_{1.5}Cu₅—Y-type | La_{1.5}Cu₅/γ-Al₂O₃ | La_{1.5}Cu₅—Y-type |
| | | molecular | | molecular sieve |
| | | sieve/γ-Al₂O₃ | | |
| HCl | 190 °C | 98.13 | 94.32 | 96.51 |
| conversion | 200 °C | 98.59 | 96.51 | 97.39 |
| rate/% | 210 °C | 99.11 | 97.42 | 98.00 |
| | 220 °C | 98.90 | 96.33 | 97.10 |
| | 230 °C | 98.87 | 96.21 | 97.02 |
| EDC | 190 °C | 97.02 | 91.33 | 94.07 |
| selectivity/% | 200 °C | 96.53 | 91.2 | 93.17 |
| | 210 °C | 96.48 | 90.72 | 92.8 |
| | 220 °C | 96.27 | 90.03 | 92.46 |
| | 230 °C | 96.08 | 89.72 | 92.11 |

In the ethylene oxychlorination catalyst of the present disclosure, γ-Al₂O₃ and a Y-type molecular sieve are used as a composite support, the active components and additives are loaded to optimize the catalytic performance. The ethylene oxychlorination catalyst has a large specific surface area, abundant pore structures, and excellent thermal stability, and can significantly enhance the conversion rate of hydrogen chloride and the selectivity for EDC. According to the examples, as the loading amount of copper increases, the Cu₅-Y-type molecular sieve/γ-Al₂O₃ composite support catalyst exhibits superior activity, and can achieve a conversion rate of 99.31% for hydrogen chloride and a selectivity of 95.03% for EDC. However, the excessively-high loading amount of copper may lead to the agglomeration of a copper species to produce large copper oxide particles, such that it fails to provide sufficient active sites, which impairs the catalytic effect. Therefore, the modification with the additive can effectively improve the performance of the catalyst, and can further improve the activity and selectivity of the catalyst, thereby increasing the efficiency of oxychlorination. The addition of the additive can enhance the dispersion of copper as an active component, and slow down the deactivation of active sites. The La_{1.5}Cu₅-Y-type molecular sieve/γ-Al₂O₃ composite support catalyst demonstrates excellent activity, and can lead to a conversion rate of 99.61% for hydrogen chloride and a selectivity of 98.71% for EDC.

The above are merely preferred embodiments of the present disclosure. It should be noted that a person of ordinary skill in the art may further make several improvements and modifications without departing from the principle of the present disclosure, but such improvements and modifications should be deemed as falling within the scope of the present disclosure.

What is claimed is:

1. An ethylene oxychlorination catalyst, comprising: a composite support and an active component attached to the composite support, wherein
the composite support is produced from γ-Al₂O₃ and a Y-type molecular sieve; and
the active component comprises copper, with a loading amount of the copper being 3 wt % to 6 wt %.

2. The ethylene oxychlorination catalyst of claim 1, wherein the active component further comprises an additive, wherein the additive comprises at least one selected from the group consisting of magnesium, cerium, calcium, potassium, and lanthanum, with a loading amount of the additive being 0.5 wt % to 2 wt %.

3. The ethylene oxychlorination catalyst of claim 1, wherein a mass ratio of the γ-Al₂O₃ to the Y-type molecular sieve is in a range of 1-2:1.

4. The ethylene oxychlorination catalyst of claim 1, wherein the Y-type molecular sieve is selected from the group consisting of a mesoporous Y-type molecular sieve and a microporous Y-type molecular sieve.

5. A method for preparing the ethylene oxychlorination catalyst of claim 1, comprising the following steps:
mixing the γ-Al₂O₃, the Y-type molecular sieve, and water, and conducting a first impregnation, a first drying, and a first calcination in sequence to obtain the composite support; and
mixing the composite support and a copper salt solution, and conducting a second impregnation, a second drying, and a second calcination in sequence to obtain the ethylene oxychlorination catalyst.

6. The method of claim 5, wherein mixing the γ-Al₂O₃, the Y-type molecular sieve, and the water is conducted through stirring and an ultrasonic treatment in sequence, and the ultrasonic treatment is conducted for 20 minutes to 40 minutes;
the first impregnation is conducted for 10 hours to 15 hours;
the first drying is conducted at a temperature of 100 °C to 130 °C; and
the first calcination is conducted at a temperature of 500 °C to 600 °C in an air atmosphere for 4 hours to 6 hours.

7. The method of claim 5, wherein a copper salt in the copper salt solution comprises at least one selected from the group consisting of copper nitrate and copper chloride; and a mass ratio of the composite support to the copper salt solution based on the copper is in a range of 1:0.03-0.06.

8. The method of claim 5, wherein mixing the composite support and the copper salt solution is conducted through stirring and an ultrasonic treatment in sequence, and the ultrasonic treatment is conducted for 20 minutes to 40 minutes;
the second impregnation is conducted for 10 hours to 15 hours;
the second drying is conducted at a temperature of 100 °C to 130 °C for 10 hours to 15 hours; and
the second calcination is conducted at a temperature of 500 °C to 600 °C in an air atmosphere for 4 hours to 6 hours.

9. A process for preparing 1,2-dichloroethane through ethylene oxychlorination, comprising the following steps:
subjecting ethylene, hydrogen chloride, and oxygen as raw materials to an oxychlorination reaction in the presence of a catalyst to obtain the 1,2-dichloroethane, wherein
the oxychlorination reaction is conducted under a pressure of 0.1 MPa; and
the catalyst is the ethylene oxychlorination catalyst of claim 1.

10. The process of claim 9, wherein a flow rate ratio of the ethylene, the hydrogen chloride, and the oxygen is in a range of 1.05-1.1:2:0.6-0.7, a total flow rate of the ethylene, the hydrogen chloride, and the oxygen is 35 mL/min to 38 mL/min, and based on the hydrogen chloride, a gas hourly space velocity is 200 h⁻¹ to 250 h⁻¹;
the oxychlorination reaction is conducted at a temperature of 190 °C to 230 °C; and
the oxychlorination reaction is conducted in a reactor, and the reactor comprises at least one selected from the group consisting of a fixed bed reactor and a fluidized bed reactor.

11. The ethylene oxychlorination catalyst of claim 3, wherein the Y-type molecular sieve is selected from the group consisting of a mesoporous Y-type molecular sieve and a microporous Y-type molecular sieve.

12. The method of claim 5, wherein the active component further comprises an additive, wherein the additive comprises at least one selected from the group consisting of magnesium, cerium, calcium, potassium, and lanthanum, with a loading amount of the additive being 0.5 wt % to 2 wt %.

13. The method of claim 5, wherein a mass ratio of the γ-Al₂O₃ to the Y-type molecular sieve is in a range of 1-2:1.

14. The method of claim 5, wherein the Y-type molecular sieve is selected from the group consisting of a mesoporous Y-type molecular sieve and a microporous Y-type molecular sieve.

15. The method of claim 7, wherein mixing the composite support and the copper salt solution is conducted through stirring and an ultrasonic treatment in sequence, and the ultrasonic treatment is conducted for 20 minutes to 40 minutes;
the second impregnation is conducted for 10 hours to 15 hours;
the second drying is conducted at a temperature of 100 °C to 130 °C for 10 hours to 15 hours; and
the second calcination is conducted at a temperature of 500 °C to 600 °C in an air atmosphere for 4 hours to 6 hours.

16. The process of claim 9, wherein the active component further comprises an additive, wherein the additive comprises at least one selected from the group consisting of magnesium, cerium, calcium, potassium, and lanthanum, with a loading amount of the additive being 0.5 wt % to 2 wt %.

17. The process of claim 9, wherein a mass ratio of the γ-Al₂O₃ to the Y-type molecular sieve is in a range of 1-2:1.

18. The process of claim 9, wherein the Y-type molecular sieve is selected from the group consisting of a mesoporous Y-type molecular sieve and a microporous Y-type molecular sieve.
